(19) United States
(12) Patent Application Publication
(10) Pub. No.: US 2012/0263709 A1
(43) Pub. Date: Oct. 18, 2012

(54) USE OF IL-33 ANTAGONISTS TO TREAT FIBROTIC DISEASES

(75) Inventors: Andrew L. Rankin, Waltham, MA (US); Stefan Pflanz, Redwood City, CA (US); John Mumm, Los Altos Hills, CA (US)

(73) Assignee: SCHERING CORPORATION, Kenilworth, NJ (US)

(21) Appl. No.: 13/394,761

(22) PCT Filed: Sep. 1, 2010
(86) PCT No.: PCT/US10/47461

§ 371 Date: Jun. 20, 2012

Related U.S. Application Data
(60) Provisional application No. 61/241,324, filed on Sep. 10, 2009.

(51) Int. Cl. A61K 39/395; A61P 1/00; A61P 11/00; A61P 17/00; A61K 38/02; A61K 31/7052

(52) U.S. Cl. 424/133.1; 424/139.1; 514/1.1; 514/44 A

(57) ABSTRACT

Methods for treating fibrotic disease, such as idiopathic pulmonary fibrosis and scleroderma, with antagonists of IL-33 are disclosed.

### FIELD OF THE INVENTION

The present invention relates generally to methods of treating fibrotic disease involving agents that antagonize IL-33.

### BACKGROUND OF THE INVENTION

Fibrosis is characterized by the excess accumulation of extracellular matrix (ECM) components including collagen. Wynn (2008) J. Pathol. 214:199-210; Sivakumar & Das (2008) Inflamm. Res. 57:410-418. Fibrosis is thought to be a consequence of chronic tissue irritation or damage. Wynn (2008) J. Pathol. 214:199-210; Friedman (2008) Gastroenterology 134:1655-1669; Trojanowska & Varga (2007) Curr. Opin. Rheumatol. 19:568-573; Selman & Pardo (2006) Proc. Am. Thorac. Soc. 3:364-372. The progressive replacement of parenchymal tissues with ECM is observed in fibrotic diseases such as systemic sclerosis, idiopathic pulmonary fibrosis and liver cirrhosis, leading to impaired organ function. Fibrosis is estimated to contribute to nearly 45% of deaths in the developed world. However, the cellular and molecular factors that sustain the fibrotic cascade remain poorly understood.

Idiopathic pulmonary fibrosis (IPF) is a non-neoplastic pulmonary disease characterized by excessive fibrosis (formation of scar tissue) in the lung. Meltzer & Noble (2008) Orphanet J. Rare Dis. 3:8. IPF is estimated to affect five million people worldwide, with a mean age at presentation of 66 years. IPF is typically progressive, with median survival of 2 to 5 years from diagnosis. Id. Lung transplant is the only effective therapy presently available. See also Mendelian Inheritance in Man No. 178500, available via the U.S. National Institutes of Health NCBI/OMIM website. The etiology of IPF is not completely understood.

Scleroderma (also referred to as systemic sclerosis, or SSc) is an autoimmune connective tissue disease characterized by extensive fibrosis of the skin and visceral organs, as well as obliteration of the lumen of small arteries. Gabrielli et al. (2007) Curr. Op. Immunol. 19:640. Progression may lead to hidebound skin and damage to the gastrointestinal tract, lungs, heart and kidneys. An estimated 300,000 people in the United States have scleroderma, with women outnumbering men 4:1, and with average age at diagnosis in their 40s. Autoantibodies against several intracellular (nuclear) antigens are commonly observed, and although their presence is useful in diagnosis, there is as yet no direct evidence linking these antibodies to the pathogenesis of the disease. There is currently no cure for scleroderma, although treatment of its various symptoms is often available.

The need exists for improved methods for treating fibrotic diseases, such as idiopathic pulmonary fibrosis and scleroderma.

### SUMMARY OF THE INVENTION

The present invention meets these needs and more by providing methods of treating fibrotic disease comprising administration of an antagonist of IL-33.

In one embodiment, the fibrosis is fibrosis of an epithelial barrier tissue, such as skin, lung or gut. In various embodiments, the fibrotic disease is selected from the group consisting of eosinophilic esophagitis, hypereosinophilic syndromes (HES), Loeffler's endomyocarditis, endomyocardial fibrosis, idiopathic pulmonary fibrosis, and scleroderma. In specific embodiments, the fibrotic disease is idiopathic pulmonary fibrosis or scleroderma.

In various embodiments, the IL-33 antagonist is selected from the group consisting of an antagonist antibody (or antigen binding fragment thereof), a small molecule, a soluble ST2 polypeptide, or a nucleic acid expression inhibitor (such as an antisense nucleic acid or an siRNA molecule). In a specific embodiment, the IL-33 antagonist is an antagonist antibody, or antigen binding fragment thereof.

### BRIEF DESCRIPTION OF THE DRAWINGS

FIGS. 1A-1D present results obtained when IL-33 is injected subcutaneously in mice, as described in greater detail at Example 3.

FIG. 1A presents photomicrographs of representative skin sections obtained from mouse serum albumin (MSA) injected- (control) or IL-33-injected (IL-33) mice stained with H&E. In this and other figures herein, the letter “A” within a photomicrograph indicates subcutaneous adipose tissue and “F” indicates a somewhat darker region of subcutaneous fibrotic tissue, which is present only in the IL-33 treated tissue. In this and other figures herein, scale bars indicate 200 μm.

FIG. 1B shows collagen content GO in dermal punches isolated from MSA- (control) or IL-33-injected mice. Symbols indicate values obtained from individual mice and lines indicate the average values throughout the figures. ***, p=0.001; Student's T-test.

FIG. 1C shows the number of eosinophils in skin sections obtained from MSA- (open circles) or IL-33-injected (closed circles) mice. ***, p=0.001; Student's T-test.

FIG. 1D shows the number of CD3⁺ cells in skin sections obtained from MSA- (control) or IL-33-injected (closed circles) mice. Experiments were repeated independently at least two times with comparable results.

FIG. 2 presents photomicrographs of representative H&E stained skin sections obtained from wild type B6/129F2 and C57B1/6 mice (top row), as well as IL-1RAcP^{-/-} B6/129F2 and ST2^{-/-} C57B1/6 mice (bottom row), all of which had been injected with IL-33. Experiments were performed with at least 5 mice per group and were repeated independently at least two times with comparable results. See Example 4.

FIGS. 3A-3D present TAQman® real time gene expression analysis of mRNA expression for various genes in skin samples obtained from MSA- (open circles) or IL-33-injected (closed circles) mice. FIG. 3A shows results for Th2 cytokines IL-4, IL-5 and IL-13. FIGS. 3B and 3C show results for various ECM-associated genes (collagen VIa, collagen IIIa, fibronectin, TIMP1, MMP-12, and MMP-13). FIG. 3D shows results for TGF-β. Individual data points represent values obtained from individual mice; lines indicate the average result. Experiments were repeated independently at least two times with comparable results. ***, p=0.008; **, p=0.016; *, p=0.032; Mann-Whitney U-test. See Example 5.

FIG. 4 provides average expression levels (and standard errors) for various extracellular matrix (ECM)-associated genes in skin samples obtained from MSA- (“Control”) or IL-33-injected (“IL-33”) mice (N=5 mice/condition). Expression levels were determined by TAQman® real time gene expression analysis. FIG. 4 also provides the ratio of expression in IL-33 KO cells compared with control cells (“Fold Δ”), and the P-value for the difference between IL-33 and Control expression levels calculated using the Mann-Whitney U-test.

FIGS. 5A-5G present results from experiments to assess the role of IL-13 in IL-33 responses, as described in greater detail at Example 6.

FIG. 5A presents photomicrographs of representative skin sections obtained from IL-33-injected wild-type (wt) or IL-13 KO (IL-13^{-/-}) mice stained with H&E.

FIG. 5B shows collagen content GO in dermal punches obtained from MSA- (control) and IL-33-treated (IL-33) wild type (WT) and IL-13^{-/-} (KO) mice. Symbols represent values obtained from individual mice and lines indicate the average values throughout the figure. ***, p<0.0001; Student's T-test.

FIG. 5C shows TAQman® real time gene expression analysis of collagen VIa and collagen IIIa genes in IL-33-treated wild type (WT) and IL-13^{-/-} (KO) mice.

FIG. 5D shows TAQman® real time gene expression analysis of MMP12, MMP13 and TIMP1 genes in IL-33-treated wild type (WT) and IL-13^{-/-} (KO) mice. ***, p=0.008; *, p=0.016 Mann-Whitney U-test.

FIG. 5E shows TAQman® real time gene expression analysis of fibronectin in individual skin samples obtained from MSA- (control) or IL-33-treated wild type (WT) and IL-13^{-/-} mice (KO).

FIG. 5F shows the number of eosinophils in skin sections obtained from IL-33-injected wild type (WT) or IL-13^{-/-} (KO) mice.

### DETAILED DESCRIPTION

All references cited herein are incorporated by reference to the same extent as if each individual publication, database entry (e.g. GenBank sequences or GeneID entries), patent application, or patent, was specifically and individually indicated to be incorporated by reference. GenBank accession numbers for nucleic acid and protein sequences referenced herein refer to the contents of the database as of the filing date of this application. Although such database entries may be subsequently modified, GenBank maintains a public record of all prior versions of the sequences as a function of date, making such database entries an unambiguous reference to a specific sequence.

This statement of incorporation by reference is intended by Applicants to relate to each and every individual publication, database entry (e.g. GenBank sequences or GeneID entries), patent application, or patent even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

### I. Definitions

As used herein, including the appended claims, the singular forms of words such as “a,” “an,” and “the,” include their corresponding plural references unless the context clearly dictates otherwise.

“Approximately,” as used herein, refers to the value of a parameter measured with the degree of precision that is typical in the art. Specifically, it refers to a value that would be within typical tolerances for such parameter, as understood by one of skill in the art.

As used herein, unless otherwise indicated (e.g. IL-33), the “mature” form of a protein refers to the polypeptide chain remaining after removal of the signal sequence (leader peptide), e.g. the form of the protein that it secreted from the cell. Signal sequences from the proteins disclosed herein are referenced in the Sequence Listing.

As used herein, “epithelial barrier tissue” refers to tissues that have regular contact with the external environment and provide a barrier function. Exemplary epithelial barrier tissues include skin, lung and gut.

As used herein, the term “antibody” may refer to any form of antibody that exhibits the desired biological activity. Thus, it is used in the broadest sense and specifically covers monoclonal antibodies (including full length monoclonal antibodies), polyclonal antibodies, multispecific antibodies (e.g., bispecific antibodies), chimeric antibodies, humanized antibodies, fully human antibodies, etc. so long as they exhibit the desired biological activity.

As used herein, when referring to antibodies, the terms “binding fragment thereof” or “antigen binding fragment thereof” encompass a fragment or a derivative of an antibody that still substantially retains the ability to bind to its target. Examples of antibody fragments include Fab, Fab', F(ab')₂, and Fv fragments; diabodies; linear antibodies; single-chain antibody molecules, e.g., sc-Fv; and multispecific antibodies formed from antibody fragments. Typically, a binding fragment or derivative retains at least 10% of its affinity for its target, e.g. no more than a 10-fold change in the dissociation equilibrium binding constant (Kd). Preferably, a binding fragment or derivative retains at least 25%, 50%, 60%, 70%, 80%, 90%, 95%, 99% or 100% (or more) of its binding affinity, although any binding fragment with sufficient affinity to exert the desired biological effect will be useful. It is also intended that, when specified, a binding fragment can include sequence variants with conservative amino acid substitutions.

“Specifically” or “selectively” binds, when referring to the binding of an antibody to its antigen, indicates a binding reaction which is determinative of the presence of the protein in a heterogeneous population of proteins and other biologics. As is commonly understood in the field, and reiterated here for emphasis, an antibody that is said to bind specifically to a polypeptide comprising a given sequence (e.g. IL-33) binds to polypeptides comprising the sequence of IL-33 but does not bind to proteins lacking the sequence of IL-33. For example, an antibody that specifically binds to a polypeptide comprising IL-33 may bind to a FLAG®-tagged form of IL-33 but will not bind to other FLAG®-tagged proteins.

The term “monoclonal antibody,” as used herein, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic epitope. In contrast, conventional (polyclonal) antibody preparations typically include a multitude of antibodies directed against (or specific for) different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. U.S. Pat. No. 4,816,567; Morrison et al. (1984) Proc. Natl. Acad. Sci. USA 81: 6851-6855.

As used herein, the term “humanized antibody” refers to forms of antibodies that contain sequences from non-human (e.g., murine) antibodies as well as sequences from human antibodies. Such antibodies contain minimal sequence derived from non-human immunoglobulin. In general, the variable domains of a humanized antibody will comprise at least one, and up to six, of the complementarity determining regions (CDR) (also referred to as hypervariable loops) of a non-human immunoglobulin, with all or substantially all of the framework (FR) regions derived from human immunoglobulin sequences.

The term “fully human antibody” refers to an antibody that comprises human immunoglobulin protein sequences only. A fully human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences, respectively. A fully human antibody may be generated in a human being, in a transgenic animal having human immunoglobulin germline sequences, by phage display or other molecular biological methods.

“IL-33” refers human interleukin-33, and is also known as aka IL-1F11, NF-HEV, and C9orf26. IL-33 is further described at online Mendelian Inheritance in Man (OMIM) entry 608678 and Gene ID No. 90865, and an exemplary naturally occurring nucleic acid and protein sequences for human IL-33 are found at GenBank Accession Nos. NM_{—}033439.2 (SEQ ID NO: 5) and AY905581 (SEQ ID NO: 6), respectively, all of which are available through the NCBI website. See also Schmitz et al. (2005) Immunity 23: 479-490. The contents of all database entries recited in the paragraph are hereby incorporated by reference in their entireties. A mature form of IL-33 comprising residues 112-270 of SEQ ID NO: 6 has been proposed to be an active form of IL-33. Schmitz et al. (2005) Immunity 23:479-490.

“ST2” as used herein is synonymous with the T1/ST2 protein, and is also known as IL-1RL1, DER-4, IL-1R4, Fit-1 and IL-1RL1. ST2 is further described at OMIM entry 601203 and Gene ID No. 9173. “Soluble ST2” (sST2) refers to a soluble extracellular variant of the membrane-bound form of ST2 (ST2L). Exemplary naturally occurring sequences for human sST2 are found at GenBank Accession Nos. NM_{—}003856.2 (SEQ ID NO: 7)/NP_{—}003847 (SEQ ID NO: 8), and exemplary naturally occurring sequences for human ST2L are found at NM_{—}016232.4 (SEQ ID NO: 9)/NP_{—}057316 (SEQ ID NO: 10), all of which are available through the NCBI website. The contents of all database entries recited in the paragraph are hereby incorporated by reference in their entireties. As is apparent from comparison of SEQ ID NOs: 8 and 10, sST2 comprises residues 1-323 of ST2L and an additional C-terminal five amino acid residues (SKECF).

“IL-1RAcP” means the interleukin-1 receptor accessory protein, is also known as IL-1R3 and IL1RAP. IL-1RAcP is further described at online OMIM entry 602626 and Gene ID No. 3556, and exemplary naturally occurring sequences for human IL-1RAcP are found at GenBank Accession Nos. NM_{—}134470.2 (SEQ ID NO: 1)/NP_{—}608273 (SEQ ID NO: 2), and at NM_{—}002182.2 (SEQ ID NO: 3)/NP_{—}002173 (SEQ ID NO: 4), all of which are available through the National Center for Biotechnology Information (NCBI) website. The contents of all database entries recited in the paragraph are hereby incorporated by reference in their entireties.

II. Treatment of Fibrotic Disease with IL-33 Antagonists

IL-33 is the most recently discovered member of the IL-1 cytokine family. Schmitz et al. (2005) Immunity 23:479-490. IL-33 is constitutively expressed in barrier tissues such as skin where it is found preferentially localized to the nucleus of epithelial and endothelial cells. Baekkevold et al. (2003) Am. J. Pathol. 163:69-79; Carriere et al. (2007) Proc. Nat'l Acad. Sci. (USA) 104:282-287; Kuchler et al. (2008) Am. J. Pathol. 173:1229-1242; Moussion et al. (2008) PLoS ONE 3:e3331. The receptor for IL-33 is composed of two subunits, IL-1RAcP and ST2. Schmitz et al. (2005) Immunity 23:479-490; Chackerian et al. (2007) J Immunol 179:2551-2555; U.S. Pat. No. 7,560,530.

IL-1RAcP is widely expressed, whereas ST2 expression is restricted to cell types that include Th2 cells, eosinophils, basophils, iNK:T cells and mast cells. Schmitz et al. (2005) Immunity 23:479-490; Lohning et al. (1998) Proc. Nat'l Acad. Sci. (USA) 95:6930-6935; Suzukawa et al. (2008) Lab. Invest. 88:1245-1253; Kondo et al. (2008) Int. Immunol. 20:791-800; Cherry et al. (2008) J. Allergy Clin. Immunol. 121:1484-1490; Smithgall (2008) Int. Immunol. 20:1019-1030. Consistent with the expression of ST2 by Th2-associated cell types, in vivo systemic administration of recombinant IL-33 induces Th2 cytokine production, eosinophilia and mucous hypersecretion in the lung and gut. Schmitz et al. (2005) Immunity 23:479-490; Kondo et al. (2008) International Immunology 20:791-800. IL-33 is released by cells undergoing necrotic cell death and in this respect IL-33 is thought to function as damage associated molecular pattern (DAMP). Cayrol & Girard (2009) Proc. Nat'l Acad. Sci. (USA) 106:9021-9026; Haraldsen et al. (2009) Trends Immunol. 30:227-233; Lüthi et al. (2009) Immunity 31: 84.

Recent studies have revealed an association between ST2 and the development of multiple diseases. A single nucleotide polymorphism in the promoter of the ST2 gene was found to associate with atopic dermatitis. Shimizu et al. (2005) Hum. Mol. Genet. 14:2919-2927. In addition, sera obtained from systemic sclerosis patients and idiopathic pulmonary fibrosis patients exhibiting acute exacerbation contained elevated levels of sST2. Kuroiwa et al. (2001) Biochem. Biophys. Res. Comm. 284:1104-1108; Tajima et al. (2003) Chest 124:1206-1214. Expression of ST2 (mRNA) was also found to be significantly increased in fibrotic liver tissue. Marvie et al. (2010) J. Cell. Mol. Med. 14(6b): 1726. Strikingly, administration of an sST2-Fc fusion protein exacerbated disease manifestations in a mouse model of liver fibrosis. Amatucci et al. (2007) J. Leukocyte Biol. 82:124; WO 2007/140205.

IL-33 has been associated with fibrotic disease. Increased expression of IL-33 message was observed in fibrotic and cirrhotic lesions adjacent to liver tumoral masses. Marvie et al. (2010) J. Cell. Mol. Med. 14(6b): 1726. In addition, administration of IL-33 limited the development of cardiac fibrosis in a mouse model of cardiac hypertrophy (Sanada et al. (2007) J. Clin. Invest. 117:1538-1549), suggesting that IL-33 could be used to treat cardiac fibrosis (U.S. Pat. App. Pub. No. 2008/0003199). Anti-IL-33 antibodies, or variants thereof, have also been proposed for the treatment of inflammatory disorders, such as asthma. WO 2008/144610.

Despite these observations, the consequences of sustained IL-33 activity in skin have not previously been reported. To determine the effects of dysregulated IL-33 signaling in skin, IL-33 was administered repeatedly subcutaneously and its effects at the injection site were monitored.

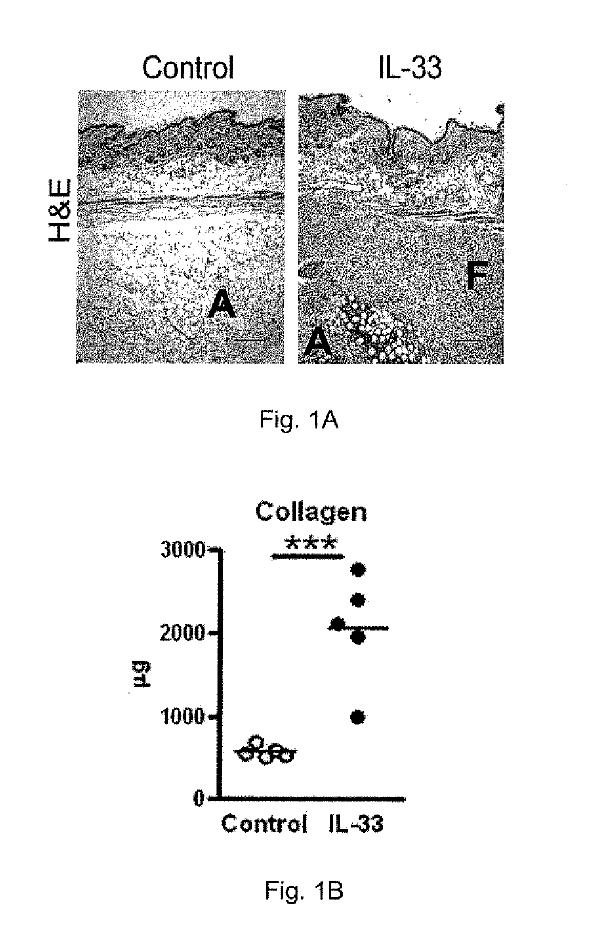

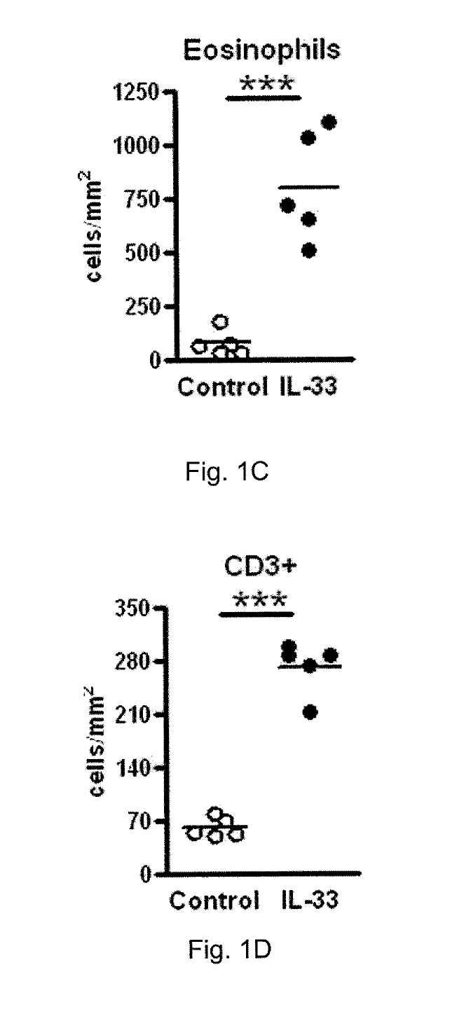

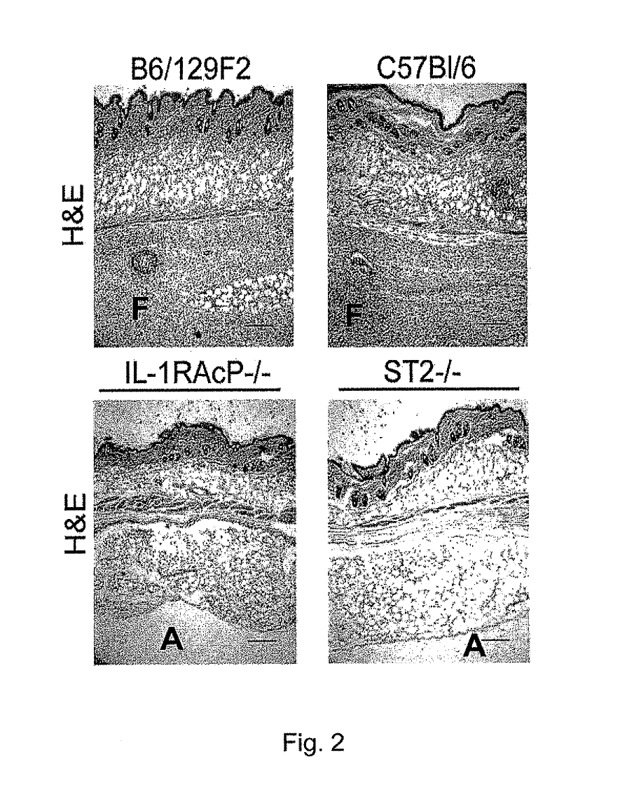

The results presented herein demonstrate that subcutaneous injection of rIL-33 can induce accumulation of extracellular matrix (collagen) and abundant inflammation comprised mainly of eosinophils, macrophages and T cells. See Example 3 and FIGS. 1-1D. These IL-33 mediated effects were ST2 and IL-1RAcP dependent (Example 4 and FIG. 2), consistent with a mechanism in which accumulation of ECM and inflammation result from IL-33 signaling via its receptor.

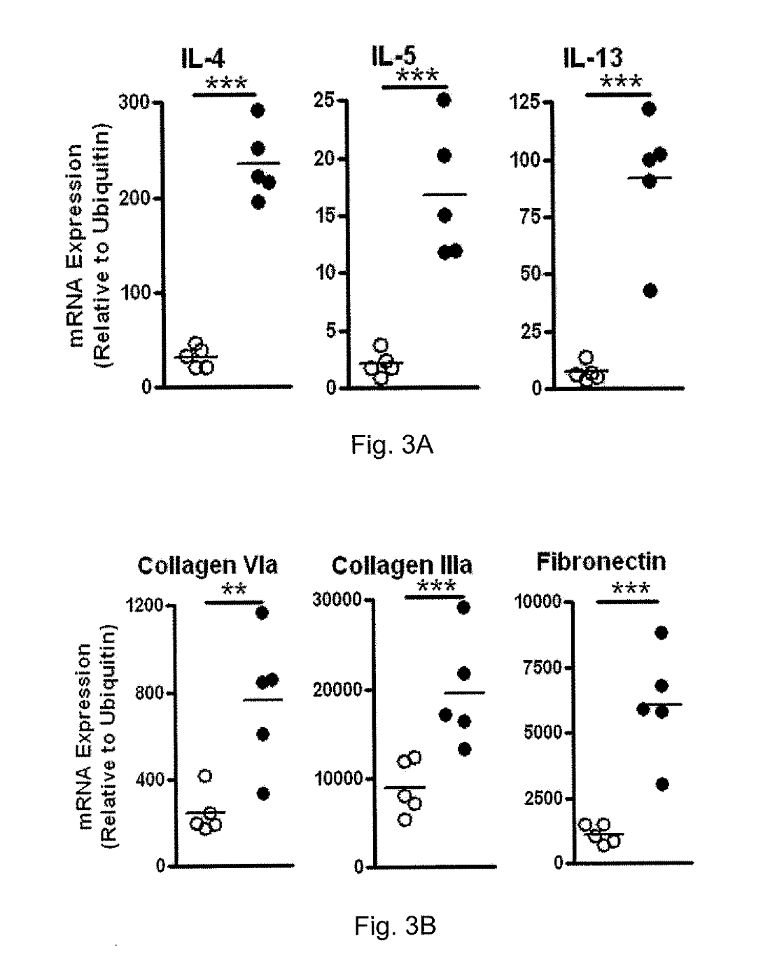

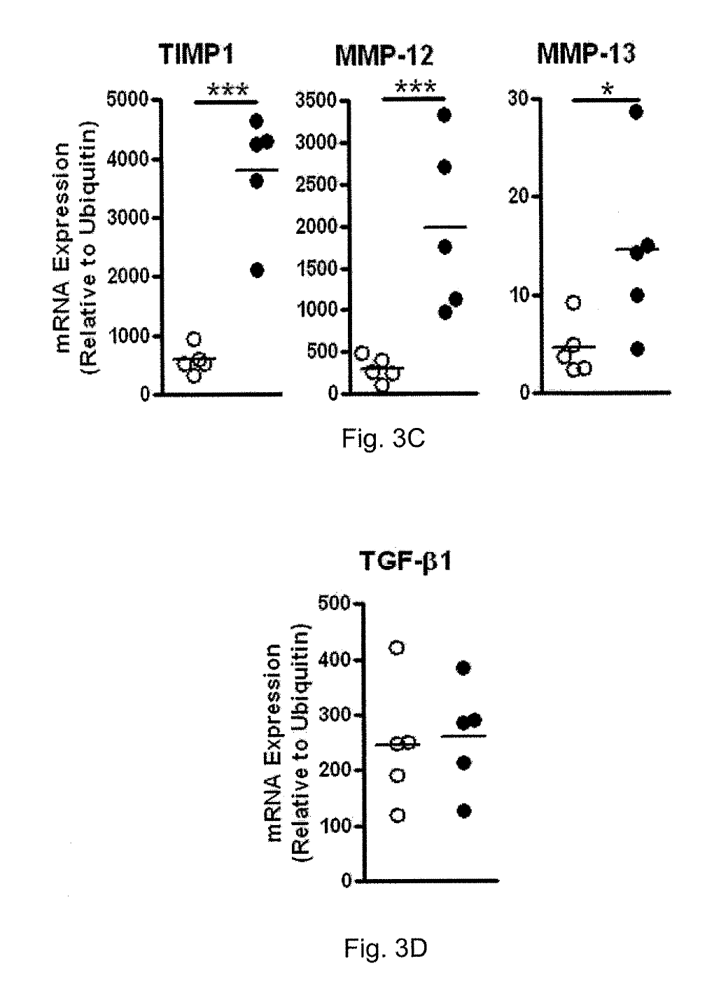

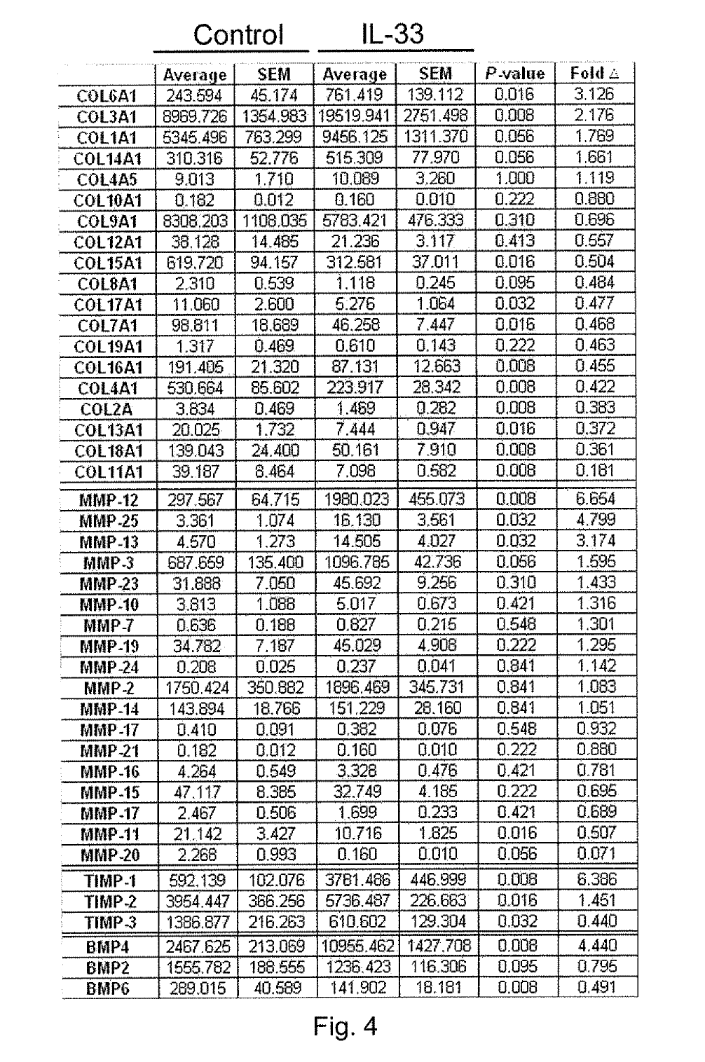

The pathologic changes induced by IL-33 were associated with altered expression of ECM genes in the collagen, MMP, TIMP and BMP families. See Example 5 and FIGS. 3A-3D and 4. In particular, the expression levels of collagen VIa and collagen IIIa were significantly increased in skin samples obtained from IL-33 injected mice suggesting that these collagen subtypes may contribute to the increased collagen deposition in IL-33 treated skin. TIMP-1 and TIMP-2 expression were also increased by IL-33 treatment. TIMP family members can inhibit MMP-mediated collagenase activity, which may promote IL-33-induced collagen accumulation by decreasing collagen turnover. Lambert et al. (2004) Crit. Rev. Oncol. Hematol. 49:187-198.

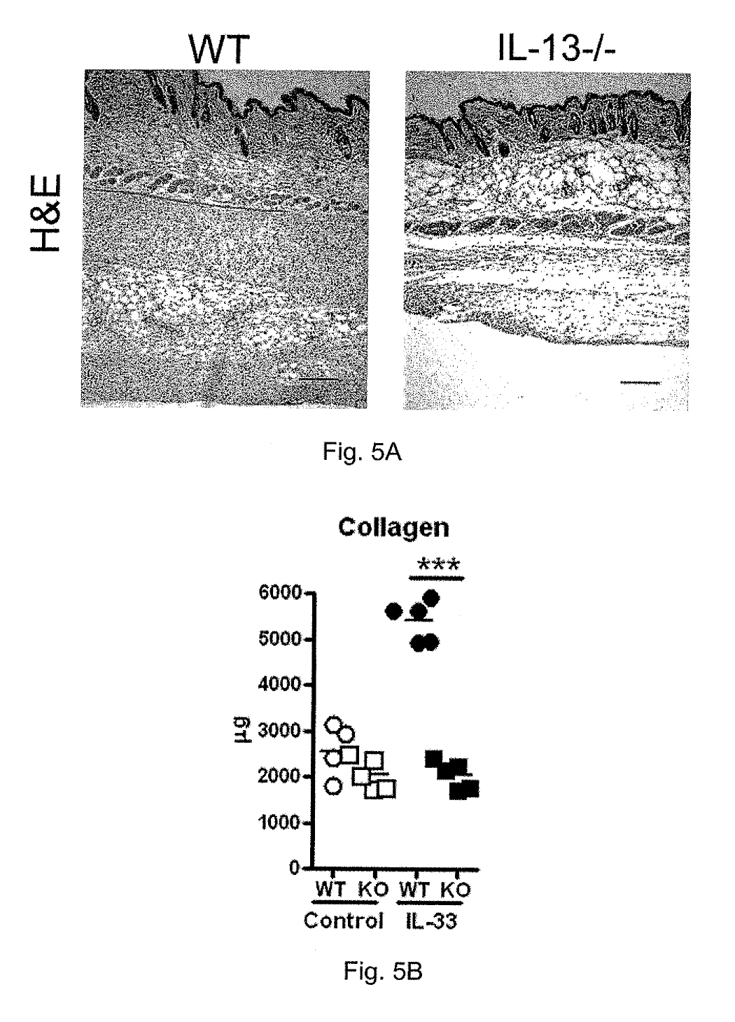

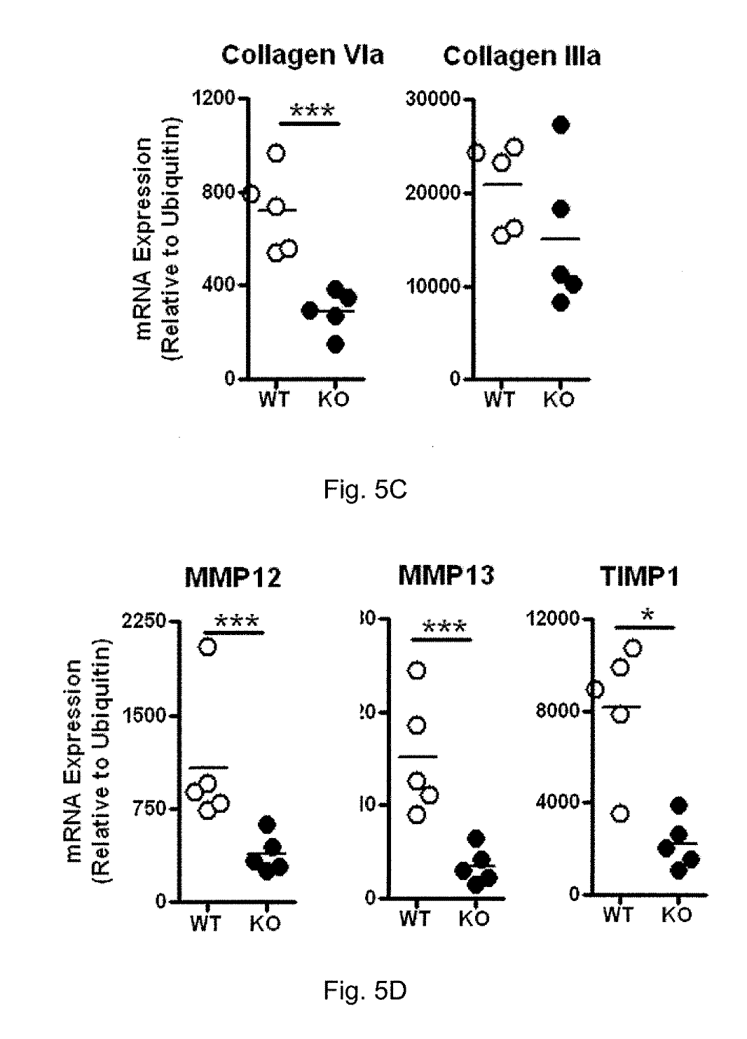

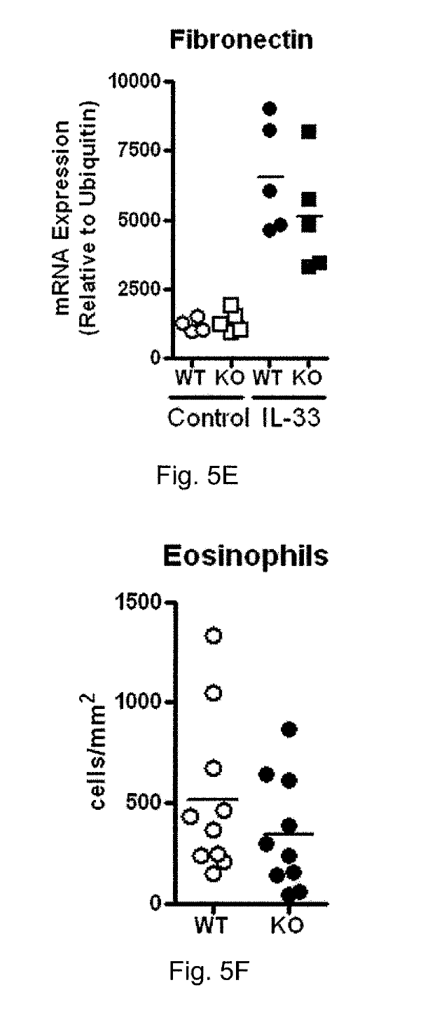

Additional data indicate that IL-33 regulates the expression of these genes through an IL-13 dependent pathway. See Example 6 and FIGS. 5A-5F. Interestingly, fibronectin-1 expression was increased independently of IL-13 in IL-33 injected skin samples, suggesting that expression of some ECM components may be regulated directly through IL-33 or other IL-13 independent downstream mediators. FIG. 5E. Overall our data highlight IL-33-mediated alterations in the expression levels of various collagen subtypes as well as enzymes known to affect ECM remodeling.

The results of the experiments presented herein establish that IL-33 induces cutaneous fibrosis and intense inflammation, and identify IL-33 as a novel factor sufficient to induce cutaneous fibrosis. These profibrotic effects were first observed in the skin of mice that received multiple subcutaneous objections of IL-33, in which the skin at the injection site thickened in IL-33 treated animals but not in controls. Experiments presented herein were then performed to further investigate the effects of dysregulated IL-33 signaling in skin. IL-33 was administered subcutaneously and its effects at the injection site were monitored. Administration of IL-33 resulted in IL-33R-dependent accumulation of eosinophils, CD3⁺ lymphocytes, F4/80⁺ mononuclear cells, increased expression of IL-13 mRNA, and the development of cutaneous fibrosis. This cutaneous fibrosis and inflammation were ST2-dependent. Consistent with extensive cutaneous tissue remodeling, IL-33 resulted in significant modulation of a number of extracellular matrix associated genes including Collagen VI, Collagen III and TIMP1. The dependence of IL-33-induced fibrosis on both IL-13 and eosinophils was established using genetically deficient mice. The data indicate that bone marrow derived eosinophils secrete IL-13 in response to IL-33 stimulation, suggesting that eosinophil derived IL-13 may promote IL-33 induced cutaneous fibrosis, i.e. that IL-13 is a critical downstream mediator of IL-33-induced cutaneous fibrosis. Collectively, these results identify IL-33 as a previously unrecognized pro-fibrotic mediator in skin and highlight the cellular and molecular pathways by which this pathology develops. The data also suggest that IL-33 may function as an endogenous DAMP involved in the initiation of fibrotic disease. See Rankin et al. (2010) J. Immunol. 184:1526.

IL-13 is a pro-fibrotic cytokine that is sufficient for the induction of fibrosis in skin and lung. Zheng et al. (2009) J. Invest. Dermatol. 129:742-751; Zhou et al. (1999) J. Clin. Invest. 103:779-788. In addition, IL-13 is required for the development of fibrosis in some animal models. Aliprantis et al. (2007) Proc. Nat'l Acad. Sci. (USA) 104:2827-2830; Liu et al. (2004) J. Immunol. 173:3425-3431; Kolodsick et al. (2004) J. Immunol. 172:4068-4076; Chiaramonte et al. (1999) J. Clin. Invest. 104:777-785. IL-13 has been shown to directly modulate expression of Collagen VIa, Collagen III and TIMP-1 in previous studies. Syed et al. (2005) Resp. Res. 6:9; Leonardi et al. (2003) Invest. Ophthalmol. & Visual Sci. 44:183-189. The results presented herein show that IL-33-induced fibrosis requires IL-13.

Abnormal accumulation of extracellular matrix is a defining characteristic of fibrotic diseases. Accordingly, in light of these results, neutralizing IL-33 activity, e.g. by antibody blockade, small molecule, or soluble receptor, may be efficacious in the treatment of fibrobtic diseases such as scleroderma and idiopathic pulmonary fibrosis. In addition, the prominent recruitment of eosinophils to sites of IL-33 injection suggests that eosinophil rich diseases, such as eosinophilic esophagitis and hypereosinophilic syndromes may benefit from blockade of IL-33. Blocking IL-33 activity in syndromes such as Loeffler's endomyocarditis and endomyocardial fibrosis may also be efficacious since these diseases exhibit both eosinophilia and fibrosis.

### III. IL-33 Antagonists

In various embodiments, the IL-33 antagonist of the present invention is an antagonist antibody (or antigen binding fragment thereof), a small molecule, a soluble ST2 polypeptide, or a nucleic acid expression inhibitor (such as an antisense nucleic acid or an siRNA molecule).

In antibody-based embodiments, the anti-IL-33 antibodies of the present invention comprises full length antibodies, or antigen binding fragments such as, but not limited to, Fab, Fab′, Fab′-SH, Fv, scFv, F(ab′)₂, and diabodies. Use of anti-IL-33 antibodies in the treatment of various inflammatory diseases, such as asthma, is disclosed at WO 2008/144610, the disclosure of which is hereby incorporated by reference in its entirety.

Any suitable method for generating monoclonal antibodies may be used. For example, a recipient may be immunized with the IL-33 or an antigenic fragment thereof. Any suitable method of immunization can be used. Such methods can include adjuvants, other immunostimulants, repeated booster immunizations, and the use of one or more immunization routes. The eliciting antigen may be a single epitope, multiple epitopes, or the entire protein alone or in combination with one or more immunogenicity enhancing agents known in the art.

Any suitable method can be used to elicit an antibody with the desired biologic property of inhibiting IL-33 activity. It is desirable to prepare monoclonal antibodies (mAbs) from various mammalian hosts, such as mice, rodents, primates, humans, etc. Techniques for preparing such monoclonal antibodies may be found in, e.g., Stites et al. (eds.) BASIC AND CLINICAL IMMUNOLOGY (4th ed.) Lange Medical Publications, Los Altos, Calif., and references cited therein; Harlow and Lane (1988) ANTIBODIES: A LABORATORY MANUAL CSH Press; Goding (1986) MONOCLONAL ANTIBODIES: PRINCIPLES AND PRACTICE (2d ed.) Academic Press, New York, N.Y. Thus, monoclonal antibodies may be obtained by a variety of techniques familiar to researchers skilled in the art. Typically, spleen cells from an animal immunized with a desired antigen are immortalized, commonly by fusion with a myeloma cell. See Kohler and Milstein (1976) Eur. J. Immunol. 6:511-519. Alternative methods of immortalization include transformation with Epstein Barr Virus, oncogenes, or retroviruses, or other methods known in the art. See, e.g., Doyle et al. (eds. 1994 and periodic supplements) CELL AND TISSUE CULTURE: LABORATORY PROCEDURES, John Wiley and Sons, New York, N.Y. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according, e.g., to the general protocol outlined by Huse et al. (1989) Science 246:1275-1281.

Other suitable techniques involve selection of libraries of antibodies in phage or similar vectors. See, e.g., Huse et al. (1989) Science 246:1275; and Ward et al. (1989) Nature 341:544. The polypeptides and antibodies of the present invention may be used with or without modification, including chimeric or humanized antibodies. Also, recombinant immunoglobulins may be produced, see Cabilly U.S. Pat. No. 4,816,567; and Queen et al. (1989) Proc. Nat'l Acad. Sci. USA 86:10029-10033; or made in transgenic mice, see Mendez et al. (1997) Nature Genetics 15:146-156; also see Abgenix and Medarex® technologies.

Also contemplated are chimeric antibodies. As noted above, typical chimeric antibodies comprise a portion of the heavy and/or light chain identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al. (1984) Proc. Natl. Acad. Sci. USA 81: 6851-6855).

Antagonists of IL-33 signaling also include antisense and interfering nucleic acid molecules, such as siRNA molecules. Antisense and siRNA molecules that reduce the expression of IL-33 may be designed based on the coding sequence for IL-33, as disclosed herein at SEQ ID NO: 5. Methods of producing and using siRNA are disclosed, e.g., at U.S. Pat. No. 6,506,559 (WO 99/32619); U.S. Pat. No. 6,673,611 (WO 99/054459); U.S. Pat. No. 7,078,196 (WO 01/75164); U.S. Pat. No. 7,071,311 and PCT publications WO 03/70914; WO 03/70918; WO 03/70966; WO 03/74654; WO 04/14312; WO 04/13280; WO 04/13355; WO 04/58940; WO 04/93788; WO 05/19453; WO 05/44981; WO 03/78097 (U.S. patents are listed with related PCT publications). Exemplary methods of using siRNA in gene silencing and therapeutic treatment are disclosed at PCT publications WO 02/096927 (VEGF and VEGF receptor); WO 03/70742 (telomerase); WO 03/70886 (protein tyrosine phosphatase type IVA (Prl3)); WO 03/70888 (Chk1); WO 03/70895 and WO 05/03350 (Alzheimer's disease); WO 03/70983 (protein kinase C alpha); WO 03/72590 (Map kinases); WO 03/72705 (cyclin D); WO 05/45034 (Parkinson's disease). Exemplary experiments relating to therapeutic uses of siRNA have also been disclosed at Zender et al. (2003) Proc. Nat'l. Acad. Sci. (USA) 100:7797; Paddison et al. (2002) Proc. Nat'l. Acad. Sci. (USA) 99:1443; and Sah (2006) Life Sci. 79:1773. siRNA molecules are also being used in clinical trials, e.g., of chronic myeloid leukemia (CML) (ClinicalTrials.gov Identifier: NCT00257647) and age-related macular degeneration (AMD) (ClinicalTrials.gov Identifier: NCT00363714).

Although the term “siRNA” is used herein to refer to molecules used to induce gene silencing via the RNA interference pathway (Fire et al. (1998) Nature 391:806), such siRNA molecules need not be strictly polyribonucleotides, and may instead contain one or more modifications to the nucleic acid to improve its properties as a therapeutic agent. Such agents are occasionally referred to as “siNA” for short interfering nucleic acids. Although such changes may formally move the molecule outside the definition of a “ribo”nucleotide, such molecules are nonetheless referred to as “siRNA” molecules herein. For example, some siRNA duplexes comprise two 19-25 nt (e.g. 21 nt) strands that pair to form a 17-23 basepair (e.g. 19 base pair) polyribonucleotide duplex with TT (deoxyribonucleotide) 3′ overhangs on each strand. Other variants of nucleic acids used to induce gene silencing via the RNA interference pathway include short hairpin RNAs (“shRNA”), for example as disclosed in U.S. Pat. App. Publication No. 2006/0115453

Soluble ST2 is believed to bind to IL-33 in solution, and thus to act as a natural antagonist of IL-33 by blocking binding to the IL-33 receptor of cell surfaces. Chackerian et al. (2007) J Immunol 179:2551-2555; Leung et al. (2004) J. Immunol. 173:145-150. Human sST2 comprises the sequence of amino acid residues 19-328 of SEQ ID NO: 8. Use of an sST2-Fc fusion protein in the treatment of lung fibrosis is disclosed at WO 2007/140205.

The broad scope of this invention is best understood with reference to the following examples, which are not intended to limit the inventions to the specific embodiments.

### EXAMPLES

### Example 1

### General Methods

Standard methods in molecular biology are described. Maniatis et al. (1982) Molecular Cloning, A Laboratory Manual, Cold Spring Harbor Laboratory Press, Cold Spring Harbor, N.Y.; Sambrook and Russell (2001) Molecular Cloning, 3^{rd} ed., Cold Spring Harbor Laboratory Press, Cold Spring Harbor, N.Y.; Wu (1993) Recombinant DNA, Vol. 217, Academic Press, San Diego, Calif. Standard methods also appear in Ausbel et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described. Coligan et al. (2000) Current Protocols in Protein Science, Vol. 1, John Wiley and Sons, Inc., New York. Chemical analysis, chemical modification, post-translational modification, production of fusion proteins, glycosylation of proteins are described. See, e.g., Coligan et al. (2000) Current Protocols in Protein Science, Vol. 2, John Wiley and Sons, Inc., New York; Ausubel et al. (2001) Current Protocols in Molecular Biology, Vol. 3, John Wiley and Sons, Inc., NY, N.Y., pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391. Production, purification, and fragmentation of polyclonal and monoclonal antibodies are described. Coligan et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, N.Y.; Harlow and Lane, supra. Standard techniques for characterizing ligand/receptor interactions are available. See, e.g., Coligan et al. (2001) Current Protocols in Immunology, Vol. 4, John Wiley, Inc., New York.

Methods for flow cytometry, including fluorescence activated cell sorting detection systems (FACS®), are available. See, e.g., Owens et al. (1994) Flow Cytometry Principles for Clinical Laboratory Practice, John Wiley and Sons, Hoboken, N.J.; Givan (2001) Flow Cytometry, 2^{nd} ed.; Wiley-Liss, Hoboken, N.J.; Shapiro (2003) Practical Flow Cytometry, John Wiley and Sons, Hoboken, N.J. Fluorescent reagents suitable for modifying nucleic acids, including nucleic acid primers and probes, polypeptides, and antibodies, for use, e.g., as diagnostic reagents, are available. Molecular Probes (2003) Catalog, Molecular Probes, Inc., Eugene, Oreg.; Sigma-Aldrich (2003) Catalog, St. Louis, Mo.

Standard methods of histology of the immune system are described. See, e.g., Muller-Harmelink (ed.) (1986) Human Thymus: Histopathology and Pathology, Springer Verlag, New York, N.Y.; Hiatt, et al. (2000) Color Atlas of Histology, Lippincott, Williams, and Wilkins, Phila., Pa.; Louis, et al. (2002) Basic Histology: Text and Atlas, McGraw-Hill, New York, N.Y.

Statistical analysis may be performed using commercially available software, including but not limited to JMP® Statistical Discovery Software, SAS Institute Inc., Cary, N.C., USA.

Cell growth media and methods are provided, e.g., at Int'l. Pat. Appl. Pub. No. WO 90/03430 and U.S. Pat. No. 5,830,761, the disclosures of which are hereby incorporated by reference in their entireties.

### Example 2

### Methods Specifically Related to the Experiments Described Herein

Animals and animal experiments were as follows. B6/129.IL-1RAcP^{-/-}, B6.IL-1R1^{-/-}, B6.RAG^{-/-} and 129S6.IL-13^{-/-} mice were housed in specific pathogen free conditions at Schering-Plough Biopharma according to IACUC guidelines. Cullinan (1998) J. Immunol. 161:5614-5620; Mombaerts et al. (1992) Cell 68:869-877; Glaccum et al. (1997) J. Immunol. 159:3364-3371; McKenzie et al. (1998) Curr. Biol. 8:339-342. B6.ST2^{-/-} mice were a kind gift from the Neurath lab, University of Mainz; Hoshino et al. (1999) J. Exp. Med. 190:1541-1547. BALB/c.Δdb1GATA, WB/B6F1.cKit^{w/v}, B6.IL-4^{-/-} and appropriate control mice were obtained from the Jackson laboratories. Yu et al. (2002) J. Exp. Med. 195:1387-1395; Metwali et al. (1996) J. Immunol. 157:4546-4553. B6/129 mice were obtained from the Jackson laboratories. C57B1/6 and 129S6 mice were obtained from Taconic. Age and sex matched mice of 8 to 16 weeks of age were used for the experiments described.

Mice were injected subcutaneously daily for 7 days with 5 μg of MSA or recombinant mIL-33. Recombinant IL-33 (AA112-270) was produced in E. coli by Aragen Biosciences and contained <0.5 EU/ml endotoxin. Twenty-four hours after the final injection the injection site was harvested for analysis. Experimental protocols were approved by the Schering-Plough Biopharma IACUC.

Quantitative RT-PCR was performed as follows. RNA isolation was performed by standard techniques and gene expression was calculated using the Δ-ΔCt method (using the mean cycle threshold value for ubiquitin and the gene of interest for each sample). Primers were obtained commercially from Applied Biosystems (Foster City, Calif.) or designed using Primer Express (PE Biosystems, Foster City, Calif.). The equation 1.8e (Ct ubiquitin−Ct gene of interest)×10⁴ was used to obtain the normalized values.

Histology was performed as follows. Skin samples from the injection site were harvested and formalin fixed for 24 hours. Samples were then paraffin embedded and 5 μm section cut. Sections were stained with H&E, modified Masson's trichrome, or astra blue and violet red according to the manufacturer's directions (American MasterTech). To quantify mast cell and eosinophil skin infiltration, astra blue and violet red stained sections were scanned using a Mirax Midi slide scanner (Zeiss). Mast cells were identified based on the characteristic astra blue staining of mast cell granules and the presence of a singular nucleus whereas eosinophils were identified by violet red cytoplasmic staining co-localized with a bi-lobed nucleus. A minimum of area of 0.5 mm² of skin per mouse was randomly selected and surveyed to obtain manual cell counts. Images were obtained using a brightfield microscope (Olympus Model BX51) with attached digital camera (Q Imaging Model Retiga 2000R) and were white balanced using Adobe Photoshop Elements (v.2.0).

Immunohistochemistry (IHC) was performed as follows. Immunohistochemical studies were performed on formalin-fixed, paraffin-embedded tissue sections using a rabbit polyclonal antibody against anti-human CD3 (Catalog #A0452, Dako Corp., dilution 1:200) and a rat monoclonal antibody against anti-human F4/80 (Clone BM8, eBioscience, dilution 1:400). Paraffin embedded tissues were sectioned at 5 μm thickness, deparaffinized and quenched with 3% hydrogen peroxide for 10 minutes. Slides were heat retrieved with Citrate Buffer at pH 6.1 (Cat. #S1699, Dako Corp.) for 4 minutes at 123° C. using the Biocare Decloaker chamber, then cooled for 15 minutes followed by a running tap water rinse. Slides were mounted on a DAKO Autostainer and covered with fresh TBS to prevent drying of sections. Sections were then incubated with anti-CD3 or anti-F4/80 antibodies at room temperature for 60 minutes and rinsed with TBS. CD3 stained sections were incubated for 30-minute in Rabbit Envision-Plus (Catalog #K4011, Dako Corp.). F4/80 stained sections were incubated for 30 minutes in Rat Probe and then Rat Polymer-HRP (Catalog #RT517L, Biocare Medical). Slides were rinsed with TBS and developed with DAB-Plus (Dako Corp.), counterstained in Modified Mayer's Hematoxylin and blued in 0.3% ammonia water followed by a tap water rinse. CD3⁻ and F4/80⁺ cells were counted as described above.

Collagen was quantified as follows. Total soluble collagen levels in the skin were quantified using the Sircol™ Assay according to the manufacturer's directions (BioColor Ltd). Briefly, a 6 mm dermal punch biopsy was isolated from the injection site and subcutaneous fat removed. Samples were homogenized in 3 ml of 0.5 M acetic acid supplemented with cOmplete™ protease inhibitors (Roche). After overnight extraction at 4° C., the samples were spun down and 0.2 ml of extract was assayed to determine collagen content. Results are reported as total micrograms of collagen extracted from 6 mm dermal punch biopsies.

Statistical analysis was performed as follows. Statistical analyses to compare collagen content in skin and cell numbers between experimental and control groups were performed using Student's t test, with p values ≦0.05 considered significant. Studies utilizing TAQman analysis to compare gene expression levels between control and experimental groups were analyzed using the Mann-Whitney U-test. p values ≦0.05 considered significant. Statistical analyses were performed using GraphPad Prism version 4.02 for Windows (GraphPad Software).

### Example 3

### IL-33 Mediated Induction of Skin Fibrosis and Inflammation

To assess the consequences of dysregulated IL-33 release in skin IL-33 or control protein (MSA) was injected subcutaneously every day for 7 days. IL-33 and MSA injected mice did not exhibit any discernible changes in behavior over the course of the injection series (e.g. pruritus) nor was any overt skin pathology observed. Nevertheless, histological analysis of skin from IL-33- but not MSA-injected mice revealed prominent inflammation and edema at the injection site (FIG. 1A) and development of subcuticular fibrosis as evidenced by Masson's Trichrome staining (not shown). Soluble collagen levels were increased approximately 4-fold in skin punch biopsies obtained from IL-33-injected mice (FIG. 1B). The majority of infiltrating leukocytes consisted of granulocytic leucocytes with dense clusters of these cells occasionally observed in subcutaneous adipose tissue (FIG. 1A). Histochemical staining revealed that the majority of infiltrating leucocytes were eosinophils and that subcutaneous injection of IL-33 caused significant accumulation of these cells compared to MSA-injected skin (FIG. 1C). Expression levels of eosinophil major basic protein in skin from IL-33 and control protein treated mice yielded a similar pattern of results (data not shown). CD3 mononuclear cells were also significantly increased in skin obtained from IL-33 injected mice (FIG. 1D) whereas mast cells and B220⁺ cells were present at similar numbers in skin sections obtained from mice injected with IL-33 or MSA (data not shown). No pathological changes were observed in skin sections adjacent to the injection site (data not shown).

### Example 4

### The Role of ST2 and IL-1RAcP in IL-33-Induced Skin Pathology

To determine if IL-33-induced inflammation is dependent on signaling through the IL-33 receptor, which is comprised of ST2 and IL-1RAcP, ST2^{-/-} and IL-1RAc^{-/-} mice were injected subcutaneously with IL-33. Neither IL-1RAc^{-/-} nor ST2^{-/-} mice exhibited histological signs of inflammation or subcuticular fibrosis after receiving IL-33 injections (FIG. 2). IL-1RAcP is a shared subunit of the IL-33R that also pairs with IL-1R1 to form the heteromeric receptor for IL-1α and IL-1β. Cullinan (1998) J. Immunol. 161:5614-5620. To determine if signaling through IL-1R1 is required for the development of IL-33-induced cutaneous fibrosis and inflammation we treated IL-1R1^{-/-} mice subcutaneously with IL-33. IL-1R1^{-/-} mice injected with IL-33 developed inflammation and subcuticular fibrosis in a manner that was indistinguishable from control mice, indicating that IL-1α/β signaling is not required for IL-33 induced skin pathology (data not shown). Collectively, these results demonstrate that repeated subcutaneous administration of IL-33 induces an ST2/IL-1RAcP-dependent skin inflammation and fibrosis.

### Example 5

### IL-33-Induced Modulation of Th2 and Extracellular Matrix-Associated Gene Expression

Gene expression in skin obtained from IL-33 injected mice was examined. IL-33-injected mice had significantly elevated expression of Th2 cytokines IL-4, IL-5 and IL-13 (FIG. 3A), whereas expression of the Th1- and Th17-associated cytokines, IFN-γ and IL-17 respectively, remained unchanged (data not shown). As repeated injection of IL-33 induced the development of subcuticular fibrosis locally, we also examined expression of a panel of extracellular matrix (ECM) associated genes (FIG. 4). IL-33-injected skin exhibited substantially increased expression of various components of the ECM, such as collagen VIa, collagen IIIa and fibronectin 1 (FIGS. 3B and 4). In addition, expression of ECM-modifying components, such as TIMP-1, MMP-12 and MMP-13 were also significantly elevated (FIG. 3C). Interestingly, IL-33 treatment also resulted in significantly reduced expression of several collagen isoforms, including collagens II, IV, XI, XIII, XVI and XVIII (FIG. 4). Thus, these results highlight the novel observation that IL-33 can modulate expression of ECM-associated genes in the skin.

TGF-β and IL-13 are two cytokines known to play a critical role in promoting fibrotic disease in multiple animal models. Wynn (2008) J. Pathol. 214:199-210. Skin isolated from IL-33 injected mice expressed levels of TGF-β that were similar to those observed in MSA-injected skin samples (FIG. 3D). Because expression of IL-13 (FIG. 3A), but not TGF-β (FIG. 3D), was significantly increased in IL-33 injected skin samples, it appears that IL-33 induced fibrosis may develop in an IL-13 dependent manner.

### Example 6

### The Role of IL-13 in IL-33 Activity

To determine if IL-33 induced skin pathology requires IL-13, IL-13^{-/-} mice were injected with IL-33 subcutaneously daily for 7 days, skin samples were examined histologically (FIG. 5A), and collagen content was quantified. Skin sections prepared from IL-13^{-/-} mice injected with IL-33 revealed only minor trichrome staining in the subcutis (data not shown), and the collagen content in skin obtained from IL-33 injected IL-13^{-/-} mice was found to be significantly reduced compared to wild type animals treated with IL-33 (FIG. 5B). Strikingly, the soluble collagen content in skin obtained from IL-33 injected IL-13^{-/-} mice was similar to levels observed in control protein treated animals, collectively indicating that IL-13 acts as a critical mediator of IL-33-induced skin fibrosis. Expression levels of Collagen VIa, Collagen IIIa, MMP12, MMP13 and TIMP1 were reduced in skin obtained from IL-33 injected IL-13^{-/-} mice compared to wild type mice (FIGS. 5C and 5D), whereas expression of fibronectin was similarly induced by IL-33 in wild type and IL-13^{-/-} mice (FIG. 5E), indicating that IL-33-dependent expression of fibronectin occurs via an IL-13 independent mechanism.

In addition, IL-33-injected IL-13^{-/-} mice exhibited a non-significant trend towards reduced eosinophil infiltrates compared with IL-33-injected wild type mice (FIG. 5F).

Table 1 provides a brief description of the sequences in the sequence listing.

TABLE 1
Sequence Identifiers

| SEQ ID NO: | Description |
|---|---|
| 1 | IL-1RAcP isoform 2 |
| | nucleic acid |
| 2 | IL-1RAcP isoform 2 |
| | polypeptide |
| 3 | IL-1RAcP isoform 1 |
| | nucleic acid |
| 4 | IL-1RAcP isoform 1 |
| | polypeptide |
| 5 | IL-33 nucleic acid |
| 6 | IL-33 polypeptide |
| 7 | ST2 isoform 2 |
| | nucleic acid |
| 8 | ST2 isoform 2 |
| | polypeptide |
| 9 | ST2 isoform 1 |
| | nucleic acid |
| 10 | ST2 isoform 1 |
| | polypeptide |

1. A method of treating a fibrotic disease in a subject comprising administering to said subject an antagonist of IL-33.

2. The method of claim 1 wherein the fibrotic disease is selected from the group consisting of eosinophilic esophagitis, hypereosinophilic syndromes (HES), Loeffler's endomyocarditis, endomyocardial fibrosis, idiopathic pulmonary fibrosis, and scleroderma.

3. The method of claim 2 wherein the fibrotic disease is idiopathic pulmonary fibrosis.

4. The method of claim 2 wherein the fibrotic disease is scleroderma.

5. The method of claim 1, wherein the antagonist of IL-33 is an antagonistic antibody, or antigen binding fragment thereof, that specifically binds to IL-33 (SEQ ID NO: 6).

6. The method of claim 5, wherein the antagonist of IL-33 is a chimeric, humanized or human antibody, or antigen binding fragment thereof.

7. The method of claim 5, wherein the antibody, or antigen binding fragment thereof, is a Fab, Fab′, F(ab′)₂, or Fv antibody fragments

8. The method of claim 1, wherein the antagonist of IL-33 is a soluble ST2 polypeptide.

9. The method of claim 1, wherein the antagonist of IL-33 is an antisense nucleic acid that specifically blocks the production of IL-33 (SEQ ID NO: 6).

10. The method of claim 1, wherein the antagonist of IL-33 is an siRNA that specifically blocks the production of IL-33 (SEQ ID NO: 6).

11. The method of claim 1, wherein the antagonist of IL-33 is a small molecule that specifically blocks the activity of IL-33.

12. The method of claim 1, wherein the antagonist blocks the binding of IL-33 to its receptor, which receptor comprises ST2 and IL-1RAcP.

13. The method of claim 1, wherein the subject is a human, and the IL-33 is human IL-33 (SEQ ID NO: 6).

14. The method of claim 1, wherein the fibrotic disease is a disease of an epithelial barrier tissue.

15. The method of claim 14, wherein the fibrotic disease is a disease of the skin.

16. The method of claim 14, wherein the fibrotic disease is a disease of the lung.

17. The method of claim 14, wherein the fibrotic disease is a disease of the gut.


SEQUENCE LISTING (10 sequences)

SEQ ID NO: 1 - DNA, 2063 bp, Homo sapiens
tgccgggatccaggtctccggggtccgctttggccagaggcgcggaaggaagcagtgcccggcgacactgcacccatcccggctgcttttgctgcgccctctcagcttcccaagaaaggcatcgtcatgtgatcatcacctaagaactagaacatcagcaggccctagaagcctcactcttgcccctccctttaatatctcaaaggatgacacttctgtggtgtgtagtgagtctctacttttatggaatcctgcaaagtgatgcctcagaacgctgcgatgactggggactagacaccatgaggcaaatccaagtgtttgaagatgagccagctcgcatcaagtgcccactctttgaacacttcttgaaattcaactacagcacagcccattcagctggccttactctgatctggtattggactaggcaggaccgggaccttgaggagccaattaacttccgcctccccgagaaccgcattagtaaggagaaagatgtgctgtggttccggcccactctcctcaatgacactggcaactatacctgcatgttaaggaacactacatattgcagcaaagttgcatttcccttggaagttgttcaaaaagacagctgtttcaattcccccatgaaactcccagtgcataaactgtatatagaatatggcattcagaggatcacttgtccaaatgtagatggatattttccttccagtgtcaaaccgactatcacttggtatatgggctgttataaaatacagaattttaataatgtaatacccgaaggtatgaacttgagtttcctcattgccttaatttcaaataatggaaattacacatgtgttgttacatatccagaaaatggacgtacgtttcatctcaccaggactctgactgtaaaggtagtaggctctccaaaaaatgcagtgccccctgtgatccattcacctaatgatcatgtggtctatgagaaagaaccaggagaggagctactcattccctgtacggtctattttagttttctgatggattctcgcaatgaggtttggtggaccattgatggaaaaaaacctgatgacatcactattgatgtcaccattaacgaaagtataagtcatagtagaacagaagatgaaacaagaactcagattttgagcatcaagaaagttacctctgaggatctcaagcgcagctatgtctgtcatgctagaagtgccaaaggcgaagttgccaaagcagccaaggtgaagcagaaaggtaatagatgcggtcagtgatgaatctctcagctccaaattaacattgtggtgaataaggacaaaaggagagattgagaacaagagagctccagcacctagcccgacggcatctaacccatagtaatgaatcaaacttaaatgaaaaatatgaaagttttcatctatgtaagatactcaaaatattgtttctgatattgttagtaccgtaatgcccaaatgtagctaaaaaaatcgacgtgagtacagtgagacacaattttgtgtctgtacaattatgaaaaattaaaaacaaagaaaatattcaaagctaccaaagatagaaaaaactggtagagccacatattgttggtgaattattaagacccttttaaaaatcattcatggtagagtttaagagtcataaaaaagattgcatcatctgacctaagactttcggaatttttcctgaacaaataacagaaagggaattatataccttttaatattattagaagcattatctgtagttgtaaaacattattaatagcagccatccaattgtatgcaactaattaaggtattgaatgtttattttccaaaaatgcataattataatattattttaaacactatgtatcaatatttaagcaggtttataatataccagcagccacaattgctaaaatgaaaatcatttaaattatgattttaaatggtataaacatgatttctatgttgatagtactatattattctacaataaatggaaattataaagccttcttgtcagaagtgctgctcctaaaaaaaaaaaaaaaaaaaaaa

SEQ ID NO: 2 - protein, 356 aa, Homo sapiens
MTLLWCVVSLYFYGILQSDASERCDDWGLDTMRQIQVFEDEPARIKCPLFEHFLKFNYSTAHSAGLTLIWYWTRQDRDLEEPINFRLPENRISKEKDVLWFRPTLLNDTGNYTCMLRNTTYCSKVAFPLEVVQKDSCFNSPMKLPVHKLYIEYGIQRITCPNVDGYFPSSVKPTITWYMGCYKIQNFNNVIPEGMNLSFLIALISNNGNYTCVVTYPENGRTFHLTRTLTVKVVGSPKNAVPPVIHSPNDHVVYEKEPGEELLIPCTVYFSFLMDSRNEVWWTIDGKKPDDITIDVTINESISHSRTEDETRTQILSIKKVTSEDLKRSYVCHARSAKGEVAKAAKVKQKGNRCGQ

SEQ ID NO: 3 - DNA, 4726 bp, Homo sapiens
tgccgggatccaggtctccggggtccgctttggccagaggcgcggaaggaagcagtgcccggcgacactgcacccatcccggctgcttttgctgcgccctctcagcttcccaagaaaggcatcgtcatgtgatcatcacctaagaactagaacatcagcaggccctagaagcctcactcttgcccctccctttaatatctcaaaggatgacacttctgtggtgtgtagtgagtctctacttttatggaatcctgcaaagtgatgcctcagaacgctgcgatgactggggactagacaccatgaggcaaatccaagtgtttgaagatgagccagctcgcatcaagtgcccactctttgaacacttcttgaaattcaactacagcacagcccattcagctggccttactctgatctggtattggactaggcaggaccgggaccttgaggagccaattaacttccgcctccccgagaaccgcattagtaaggagaaagatgtgctgtggttccggcccactctcctcaatgacactggcaactatacctgcatgttaaggaacactacatattgcagcaaagttgcatttcccttggaagttgttcaaaaagacagctgtttcaattcccccatgaaactcccagtgcataaactgtatatagaatatggcattcagaggatcacttgtccaaatgtagatggatattttccttccagtgtcaaaccgactatcacttggtatatgggctgttataaaatacagaattttaataatgtaatacccgaaggtatgaacttgagtttcctcattgccttaatttcaaataatggaaattacacatgtgttgttacatatccagaaaatggacgtacgtttcatctcaccaggactctgactgtaaaggtagtaggctctccaaaaaatgcagtgccccctgtgatccattcacctaatgatcatgtggtctatgagaaagaaccaggagaggagctactcattccctgtacggtctattttagttttctgatggattctcgcaatgaggtttggtggaccattgatggaaaaaaacctgatgacatcactattgatgtcaccattaacgaaagtataagtcatagtagaacagaagatgaaacaagaactcagattttgagcatcaagaaagttacctctgaggatctcaagcgcagctatgtctgtcatgctagaagtgccaaaggcgaagttgccaaagcagccaaggtgaagcagaaagtgccagctccaagatacacagtggaactggcttgtggttttggagccacagtcctgctagtggtgattctcattgttgtttaccatgtttactggctagagatggtcctattttaccgggctcattttggaacagatgaaaccattttagatggaaaagagtatgatatttatgtatcctatgcaaggaatgcggaagaagaagaatttgtattactgaccctccgtggagttttggagaatgaatttggatacaagctgtgcatctttgaccgagacagtctgcctgggggaattgtcacagatgagactttgagcttcattcagaaaagcagacgcctcctggttgttctaagccccaactacgtgctccagggaacccaagccctcctggagctcaaggctggcctagaaaatatggcctctcggggcaacatcaacgtcattttagtacagtacaaagctgtgaaggaaacgaaggtgaaagagctgaagagggctaagacggtgctcacggtcattaaatggaaaggggaaaaatccaagtatccacagggcaggttctggaagcagctgcaggtggccatgccagtgaagaaaagtcccaggcggtctagcagtgatgagcagggcctctcgtattcatctttgaaaaatgtatgaaaggaataatgaaaagggtaaaaagaacaaggggtgctccaggaagaaagagtccccccagtcttcattcgcagtttatggtttcataggcaaaaataatggtctaagcctcccaatagggataaatttagggtgactgtgtggctgactattctgcttcctcaggcaacactaaagtttagaaagatatcatcaacgttctgtcaccagtctctgatgccactatgttctttgcaggcaaagacttgttcaatgcgaatttccccttctacattgtctatccctgtttttatatgtctccattctttttaaaatcttaacatatggagcagcctttcctatgaatttaaatatgcctttaaaataagtcactgttgacagggtcatgagtttccgagtatagttttctttttatcttatttttactcgtccgttgaaaagataatcaaggcctacattttagctgaggataatgaacttttttcctcattcggctgtataatacataaccacagcaagactgacatccacttaggatgatacaaagcagtgtaactgaaaatgtttcttttaattgatttaaaggacttgtcttctataccacccttgtcctcatctcaggtaatttatgaaatctatgtaaacttgaaaaatatttcttaatttttgtttttgctccagtcaattcctgattatccacaggtcaacccacattttttcattccttctccctatctgcttatatcgcattgctcatttagagtttgcaggaggctccatactaggttcagtctgaaagaaatctcctaatggtgctatagagagggaggtaacagaaagactcttttagggcatttttctgactcatgaaaagagcacagaaaaggatgtttggcaatttgtcttttaagtcttaaccttgctaatgtgaatactgggaaagtgattttttctcactcgtttttgttgctccattgtaaagggcggaggtcagtcttagtggccttgagagttgcttttggcattaatattctaagagaattaactgtatttcctgtcacctattcactagtgcaggaaatatacttgctccaaataagtcagtatgagaagtcactgtcaatgaaagttgttttgtttgttttcagtaatattttgctgtttttaagacttggaaaactaagtgcagagtttacagagtggtaaatatctatgttacatgtagattatacatatatatacacacgtgtatatgagatatatatcttatatctccacaaacacaaattatatatatacatatccacacacatacattacatatatctgtgtatataaatccacatgcacatgaaatatatatatatatataatttgtgtgtgtgtatgtgtatgtatatgactttaaatagctatgggtacaatattaaaaaccactggaactcttgtccagtttttaaattatgtttttactggaatgtttttgtgtcagtgttttctgtacatattatttgttaattcacagctcacagagtgatagttgtcatagttcttgccttccctaagtttatataaataacttaagtattgctacagtttatctaggttgcagtggcatctgctgtgcacagagcttccatggtcactgctaagcagtagccagccatcgggcattaattgatttcctactatattcccagcagacacatttagaaactaagctatgttaacctcagtgctcaactatttgaactgttgagtgataaaggaaacaaatataactgtaaatgaatcttggtatcctgtgaaacagaataattcgtaatttaagaaagcccttatcccggtaacatgaatgttgatgaacaaatgtaaaattatatcctatatttaagtacccataataaatcatttccctctataagtgttattgattattttaaattgaaaaaagtttcacttggatgaaaaaagtagaaaagtaggtcattcttggatctacttttttttagccttattaatatttttccctattagaaaccacaattactccctctattaacccttcacttactagaccagaaaagaacttattccagataagctttgaatatcaattcttacataaactttaggcaaacagggaatagtctagtcaccaaaggaccattctcttgccaatgctgcattccttttgcacttttggattccatatttatcccaaatgctgttgggcacccctagaaataccttgatgttttttctatttatatgcctgcctttggtacttaattttacaaatgctgtaatataaagcatatcaagtttatgtgatacgtatcattgcaagagaatttgtttcaagatttttttttaatgttccagaagatggccaatagagaacattcaagggaaatggggaaacataatttagagaacaagaacaaaccatgtctcaaatttttttaaaaaaaattaatggttttaaatatatgctatagggacgttccatgcccaggttaacaaagaactgtgatatatagagtgtctaattacaaaatcatatacgatttatttaattctcttctgtattgtaacttagatgattcccaaggactctaataaaaaatcacttcattgtatttggaaacaaaaacatcattcattaattacttattttctttccataggttttaatattttgagagtgtcttttttatttcattcatgaacttttgtatttttcatttttcatttgatttgtaaatttacttatgttaaaaataaaccatttattttcagctttg

SEQ ID NO: 4 - protein, 570 aa, Homo sapiens
MTLLWCVVSLYFYGILQSDASERCDDWGLDTMRQIQVFEDEPARIKCPLFEHFLKFNYSTAHSAGLTLIWYWTRQDRDLEEPINFRLPENRISKEKDVLWFRPTLLNDTGNYTCMLRNTTYCSKVAFPLEVVQKDSCFNSPMKLPVHKLYIEYGIQRITCPNVDGYFPSSVKPTITWYMGCYKIQNFNNVIPEGMNLSFLIALISNNGNYTCVVTYPENGRTFHLTRTLTVKVVGSPKNAVPPVIHSPNDHVVYEKEPGEELLIPCTVYFSFLMDSRNEVWWTIDGKKPDDITIDVTINESISHSRTEDETRTQILSIKKVTSEDLKRSYVCHARSAKGEVAKAAKVKQKVPAPRYTVELACGFGATVLLVVILIVVYHVYWLEMVLFYRAHFGTDETILDGKEYDIYVSYARNAEEEEFVLLTLRGVLENEFGYKLCIFDRDSLPGGIVTDETLSFIQKSRRLLVVLSPNYVLQGTQALLELKAGLENMASRGNINVILVQYKAVKETKVKELKRAKTVLTVIKWKGEKSKYPQGRFWKQLQVAMPVKKSPRRSSSDEQGLSYSSLKNV

SEQ ID NO: 5 - DNA, 2644 bp, Homo sapiens
caacagaatactgaaaaatgaagcctaaaatgaagtattcaaccaacaaaatttccacagcaaagtggaagaacacagcaagcaaagccttgtgtttcaagctgggaaaatcccaacagaaggccaaagaagtttgccccatgtactttatgaagctccgctctggccttatgataaaaaaggaggcctgttactttaggagagaaaccaccaaaaggccttcactgaaaacaggtagaaagcacaaaagacatctggtactcgctgcctgtcaacagcagtctactgtggagtgctttgcctttggtatatcaggggtccagaaatatactagagcacttcatgattcaagtatcacaggaatttcacctattacagagtatcttgcttctctaagcacatacaatgatcaatccattacttttgctttggaggatgaaagttatgagatatatgttgaagacttgaaaaaagatgaaaagaaagataaggtgttactgagttactatgagtctcaacacccctcaaatgaatcaggtgacggtgttgatggtaagatgttaatggtaaccctgagtcctacaaaagacttctggttgcatgccaacaacaaggaacactctgtggagctccataagtgtgaaaaaccactgccagaccaggccttctttgtccttcataatatgcactccaactgtgtttcatttgaatgcaagactgatcctggagtgtttataggtgtaaaggataatcatcttgctctgattaaagtagactcttctgagaatttgtgtactgaaaatatcttgtttaagctctctgaaacttagttgatggaaacctgtgagtcttgggttgagtacccaaatgctaccactggagaaggaatgagagataaagaaagagacaggtgacatctaagggaaatgaagagtgcttagcatgtgtggaatgttttccatattatgtataaaaatattttttctaatcctccagttattcttttatttccctctgtataactgcatcttcaatacaagtatcagtatattaaatagggtattggtaaagaaacggtcaacattctaaagagatacagtctgacctttacttttctctagtttcagtccagaaagaacttcatatttagagctaaggccactgaggaaagagccatagcttaagtctctatgtagacagggatccattttaaagagctacttagagaaataattttccacagttccaaacgataggctcaaacactagagctgctagtaaaaagaagaccagatgcttcacagaattatcattttttcaactggaataaaacaccaggtttgtttgtagatgtcttaggcaacactcagagcagatctcccttactgtcaggggatatggaacttcaaaggcccacatggcaagccaggtaacataaatgtgtgaaaaagtaaagataactaaaaaatttagaaaaataaatccagtatttgtaaagtgaataacttcatttctaattgtttaatttttaaaattctgatttttatatattgagtttaagcaaggcattcttacacgaggaagtgaagtaaattttagttcagacataaaatttcacttattaggaatatgtaacatgctaaaacttttttttttttaaagagtactgagtcacaacatgttttagagcatccaagtaccatataatccaactatcatggtaaggccagaaatcttctaacctaccagagcctagatgagacaccgaattaacattaaaatttcagtaactgactgtccctcatgtccatggcctaccatcccttctgaccctggcttccagggacctatgtcttttaatactcactgtcacattgggcaaagttgcttctaatccttatttcccatgtgcacaagtctttttgtattccagcttcctgataacactgcttactgtggaatattcatttgacatctgtctcttttcatttcttttaactaccatgcccttgatatatcttttgcacctgctgaacttcatttctgtatcacctgacctctggatgccaaaacgtttattctgctttgtctgttgtagaattttagataaagctattaatggcaatatttttttgctaaacgtttttgttttttactgtcactagggcaataaaatttatactcaaccatataataacattttttaactactaaaggagtagtttttattttaaagtcttagcaatttctattacaacttttcttagacttaacacttatgataaatgactaacatagtaacagaatctttatgaaatatgaccttttctgaaaatacatacttttacatttctactttattgagacctattagatgtaagtgctagtagaatataagataaaagaggctgagaattaccatacaagggtattacaactgtaaaacaatttatctttgtttcattgttctgtcaataattgttaccaaagagataaaaataaaagcagaatgtatatcatcccatctgaaaaacactaattattgacatgtgcatctgtacaataaacttaaaatgattattaaataatcaaatatatctactacattgtttatattattgaataaagtatattttccaaatgt

SEQ ID NO: 6 - protein, 270 aa, Homo sapiens
MKPKMKYSTNKISTAKWKNTASKALCFKLGKSQQKAKEVCPMYFMKLRSGLMIKKEACYFRRETTKRPSLKTGRKHKRHLVLAACQQQSTVECFAFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 7 - DNA, 2542 bp, Homo sapiens
gaggagggacctacaaagactggaaactattcttagctccgtcactgactccaagttcatcccctctgtctttcagtttggttgagatataggctactcttcccaactcagtcttgaagagtatcaccaactgcctcatgtgtggtgaccttcactgtcgtatgccagtgactcatctggagtaatctcaacaacgagttaccaatacttgctcttgattgataaacagaatggggttttggatcttagcaattctcacaattctcatgtattccacagcagcaaagtttagtaaacaatcatggggcctggaaaatgaggctttaattgtaagatgtcctagacaaggaaaacctagttacaccgtggattggtattactcacaaacaaacaaaagtattcccactcaggaaagaaatcgtgtgtttgcctcaggccaacttctgaagtttctaccagctgcagttgctgattctggtatttatacctgtattgtcagaagtcccacattcaataggactggatatgcgaatgtcaccatatataaaaaacaatcagattgcaatgttccagattatttgatgtattcaacagtatctggatcagaaaaaaattccaaaatttattgtcctaccattgacctctacaactggacagcacctcttgagtggtttaagaattgtcaggctcttcaaggatcaaggtacagggcgcacaagtcatttttggtcattgataatgtgatgactgaggacgcaggtgattacacctgtaaatttatacacaatgaaaatggagccaattatagtgtgacggcgaccaggtccttcacggtcaaggatgagcaaggcttttctctgtttccagtaatcggagcccctgcacaaaatgaaataaaggaagtggaaattggaaaaaacgcaaacctaacttgctctgcttgttttggaaaaggcactcagttcttggctgccgtcctgtggcagcttaatggaacaaaaattacagactttggtgaaccaagaattcaacaagaggaagggcaaaatcaaagtttcagcaatgggctggcttgtctagacatggttttaagaatagctgacgtgaaggaagaggatttattgctgcagtacgactgtctggccctgaatttgcatggcttgagaaggcacaccgtaagactaagtaggaaaaatccaagtaaggagtgtttctgagactttgatcacctgaactttctctagcaagtgtaagcagaatggagtgtggttccaagagatccatcaagacaatgggaatggcctgtgccataaaatgtgcttctcttcttcgggatgttgtttgctgtctgatctttgtagactgttcctgtttgctgggagcttctctgctgcttaaattgttcgtcctcccccactccctcctatcgttggtttgtctagaacactcagctgcttctttggtcatccttgttttctaactttatgaactccctctgtgtcactgtatgtgaaaggaaatgcaccaacaaccgtaaactgaacgtgttcttttgtgctcttttataacttgcattacatgttgtaagcatggtccgttctatacctttttctggtcataatgaacactcattttgttagcgagggtggtaaagtgaacaaaaaggggaagtatcaaactactgccatttcagtgagaaaatcctaggtgctactttataataagacatttgttaggccattcttgcattgatataaagaaatacctgagactgggtgatttatatgaaaagaggtttaattggctcacagttctgcaggctgtatgggaagcatggcggcatctgcttctggggacacctcaggagctttactcatggcagaaggcaaagcaaaggcaggcacttcacacagtaaaagcaggagcgagagagaggtgccacactgaaacagccagatctcatgagaagtcactcactattgcaaggacagcatcaaagagatggtgctaaaccattcatgatgaactcacccccatgatccaatcacctcccaccaggctccacctcgaatactggggattaccattcagcatgagatttgggcaggaacacagacccaaaccataccacacacattatcattgttaaactttgtaaagtatttaaggtacatggaacacacgggaagtctggtagctcagcccatttctttattgcatctgttattcaccatgtaattcaggtaccacgtattccagggagcctttcttggccctcagtttgcagtatacacactttccaagtactcttgtagcatcctgtttgtatcatagcactggtcacattgccttacctaaatctgtttgacagtctgctcaacacgactgcaagctccatgagggcagggacatcatctcttccatctttgggtccttagtgcaatacctggcagctagccagtgctcagctaaatatttgttgactgaataaatgaatgcacaaccaaaaaaaaaaaaaaaaaaaaaaaaaaaaaaaaaaaaa

SEQ ID NO: 8 - protein, 328 aa, Homo sapiens
  SIGNAL (1)..(18)
  CHAIN (19)..(328)
MGFWILAILTILMYSTAAKFSKQSWGLENEALIVRCPRQGKPSYTVDWYYSQTNKSIPTQERNRVFASGQLLKFLPAAVADSGIYTCIVRSPTFNRTGYANVTIYKKQSDCNVPDYLMYSTVSGSEKNSKIYCPTIDLYNWTAPLEWFKNCQALQGSRYRAHKSFLVIDNVMTEDAGDYTCKFIHNENGANYSVTATRSFTVKDEQGFSLFPVIGAPAQNEIKEVEIGKNANLTCSACFGKGTQFLAAVLWQLNGTKITDFGEPRIQQEEGQNQSFSNGLACLDMVLRIADVKEEDLLLQYDCLALNLHGLRRHTVRLSRKNPSKECF

SEQ ID NO: 9 - DNA, 2058 bp, Homo sapiens
aaagagaggctggctgttgtatttagtaaagctataaagctgtaagagaaattggctttctgagttgtgaaactgtgggcagaaagttgaggaagaaagaactcaagtacaacccaatgaggttgagatataggctactcttcccaactcagtcttgaagagtatcaccaactgcctcatgtgtggtgaccttcactgtcgtatgccagtgactcatctggagtaatctcaacaacgagttaccaatacttgctcttgattgataaacagaatggggttttggatcttagcaattctcacaattctcatgtattccacagcagcaaagtttagtaaacaatcatggggcctggaaaatgaggctttaattgtaagatgtcctagacaaggaaaacctagttacaccgtggattggtattactcacaaacaaacaaaagtattcccactcaggaaagaaatcgtgtgtttgcctcaggccaacttctgaagtttctaccagctgcagttgctgattctggtatttatacctgtattgtcagaagtcccacattcaataggactggatatgcgaatgtcaccatatataaaaaacaatcagattgcaatgttccagattatttgatgtattcaacagtatctggatcagaaaaaaattccaaaatttattgtcctaccattgacctctacaactggacagcacctcttgagtggtttaagaattgtcaggctcttcaaggatcaaggtacagggcgcacaagtcatttttggtcattgataatgtgatgactgaggacgcaggtgattacacctgtaaatttatacacaatgaaaatggagccaattatagtgtgacggcgaccaggtccttcacggtcaaggatgagcaaggcttttctctgtttccagtaatcggagcccctgcacaaaatgaaataaaggaagtggaaattggaaaaaacgcaaacctaacttgctctgcttgttttggaaaaggcactcagttcttggctgccgtcctgtggcagcttaatggaacaaaaattacagactttggtgaaccaagaattcaacaagaggaagggcaaaatcaaagtttcagcaatgggctggcttgtctagacatggttttaagaatagctgacgtgaaggaagaggatttattgctgcagtacgactgtctggccctgaatttgcatggcttgagaaggcacaccgtaagactaagtaggaaaaatccaattgatcatcatagcatctactgcataattgcagtatgtagtgtatttttaatgctaatcaatgtcctggttatcatcctaaaaatgttctggattgaggccactctgctctggagagacatagctaaaccttacaagactaggaatgatggaaagctctatgatgcttatgttgtctacccacggaactacaaatccagtacagatggggccagtcgtgtagagcactttgttcaccagattctgcctgatgttcttgaaaataaatgtggctataccttatgcatttatgggagagatatgctacctggagaagatgtagtcactgcagtggaaaccaacatacgaaagagcaggcggcacattttcatcctgacccctcagatcactcacaataaggagtttgcctacgagcaggaggttgccctgcactgtgccctcatccagaacgacgccaaggtgatacttattgagatggaggctctgagcgagctggacatgctgcaggctgaggcgcttcaggactccctccagcatcttatgaaagtacaggggaccatcaagtggagggaggaccacattgccaataaaaggtccctgaattctaaattctggaagcacgtgaggtaccaaatgcctgtgccaagcaaaattcccagaaaggcctctagtttgactcccttggctgcccagaagcaatagtgcctgctgtgatgtgcaaaggcatctgagtttgaagctttcctgacttctcctagctggcttatgcccctgcactgaagtgtgaggagcaggaatattaaagggattcaggcctc

SEQ ID NO: 10 - protein, 556 aa, Homo sapiens
  SIGNAL (1)..(18)
  CHAIN (19)..(556)
MGFWILAILTILMYSTAAKFSKQSWGLENEALIVRCPRQGKPSYTVDWYYSQTNKSIPTQERNRVFASGQLLKFLPAAVADSGIYTCIVRSPTFNRTGYANVTIYKKQSDCNVPDYLMYSTVSGSEKNSKIYCPTIDLYNWTAPLEWFKNCQALQGSRYRAHKSFLVIDNVMTEDAGDYTCKFIHNENGANYSVTATRSFTVKDEQGFSLFPVIGAPAQNEIKEVEIGKNANLTCSACFGKGTQFLAAVLWQLNGTKITDFGEPRIQQEEGQNQSFSNGLACLDMVLRIADVKEEDLLLQYDCLALNLHGLRRHTVRLSRKNPIDHHSIYCIIAVCSVFLMLINVLVIILKMFWIEATLLWRDIAKPYKTRNDGKLYDAYVVYPRNYKSSTDGASRVEHFVHQILPDVLENKCGYTLCIYGRDMLPGEDVVTAVETNIRKSRRHIFILTPQITHNKEFAYEQEVALHCALIQNDAKVILIEMEALSELDMLQAEALQDSLQHLMKVQGTIKWREDHIANKRSLNSKFWKHVRYQMPVPSKIPRKASSLTPLAAQKQ